(19) United States
(12) Patent Application Publication
(10) Pub. No.: US 2016/0137963 A1
(43) Pub. Date: May 19, 2016

(54) A Microfluidic Device with a Diffusion Barrier

(71) Applicant: DANMARKS TEKNISKE UNIVERSITET, Kgs. Lyngby (DK)

(72) Inventors: Johan Eriksen, Frederiksberg (DK); Rudolphe Marie, Copenhagen V. (DK); Anders Kristensen, Frederiksberg C (DK)

(21) Appl. No.: 14/899,947

(22) PCT Filed: Jun. 30, 2014
(86) PCT No.: PCT/EP2014/063825

§ 371 Date: Dec. 18, 2015

(30) Foreign Application Priority Data
Jun. 28, 2013 (EP) 13174381.7

(51) Int. Cl. C12M 3/06; C12M 1/34; C12M 1/00; C12P 19/34; C12M 1/40

(52) U.S. Cl. CPC C12M 23/16; C12P 19/34; C12M 21/18; C12M 23/58; C12M 23/34; C12M 41/40

(57) ABSTRACT

The invention provides a microfluidic device for macromolecule amplification by sequential addition of liquid reagents. The device of the invention comprises a chip forming a plurality of reaction chambers each extending between an inlet and an outlet, each inlet being in fluid communication with a common junction via micro channels. To enable amplification of DNA, e.g. by MDA, the device comprises a diffusion barrier at each inlet configured to increase the pressure threshold for a reagent to cross the resistor. The invention further provides a method of mixing liquid reagents by use of the device where single DNA molecules are allowed to cross the diffusion barrier individually.

### INTRODUCTION

The present invention relates to a microfluidic device for performing sequential addition of liquid reagents. Particularly, the invention relates to a device comprising a chip forming a plurality of reaction chambers each extending between an inlet and an outlet.

### BACKGROUND

Sequential addition of liquid reagents is used for example in DNA amplification by extraction of DNA from a sample

So called Lab-on-a-chip technology today provides the sensitivity required to analyse single cells. This technology is typically applied where single-cell analysis should be streamlined or automated to provide a high throughput.

The term chip when used herein, relates to the down-scaling the laboratory to one single and very small element compared with a laboratory in traditional sense. Typically, the overall size is in the mm to cm regime. This has several advantages including for example less reagent volume, less reaction time, integrated processes, and the option to use the LOC for new applications. The term chip should therefore not be confused with the traditional silicon chip consisting of an integrated electrical circuit.

A microfluidic device is known e.g. from EP0843734 which discloses fluid samples which are moved from one reaction chamber to another chamber via fluid channels by applying a positive pressure differential from the originating chamber.

On-chip amplification of DNA is used for many purposes. A common amplification technique is the polymerase chain reaction, PCR which is a method for producing a large number of copies of a certain DNA fragment. While the purpose of PCR is to amplify only a fragment of the DNA, another technique for amplification of DNA is the multiple displacement amplification, MDA. Contrary to PCR, MDA amplifies the entire DNA genome. The protocol has most recently been used in a microfluidic device for amplifying DNA from single cells.

Since the first development of the LOC concept in the 1980's there has been significant improvements in functionality and fabrication techniques. However, miniaturization and commercialization of the LOC devices have been delayed e.g. due to the lack of integration of vital components such as micro pumps and micro valves.

The reaction chambers of the devices incorporate pressure inlets connecting the reaction chamber to a pressure source. Application of the pressure differential to a particular reaction chamber is carried out by selectively lowering the pressure in the receiving chamber. For this purpose, the pressure inlet for the reaction chambers is made with a controllable valve structure which is selectively operable. Application of the pressure source to the sample chamber then forces the sample into the next reaction chamber which is at a lower pressure.

The known chambers and means for pressurizing appear unsuitable for macromolecules such as genomic DNA or enzymes (proteins) in or out of the reaction chambers.

### DESCRIPTION OF THE INVENTION

To enable a simple chip structure e.g. for use in DNA amplification and particularly for use in MDA, and particularly to reduce manufacturing costs, the invention provides a microfluidic device for macromolecule amplification by performing sequential addition of liquid reagents, the device comprises, a chip forming a plurality of reaction chambers each extending between an inlet and an outlet. Each inlet is in fluid communication with a common junction, also referred to herein as a bus.

The device further comprises a diffusion barrier at each inlet. The diffusion barrier forms a resistor and it is configured to increase the pressure threshold for a reagent to cross the resistor. The fluid resistance thereby becomes higher at the resistor than before and after the resistor.

The device may e.g. be used in combination with, or it may include a pressure control structure configured to apply a control pressure at least at each outlet and at the common junction. The control pressures are applied individually at each location thereby facilitating control of fluid flow from the junction through each chamber individually. Due to the diffusion barrier, the invention provides a very simple device for macromolecule amplification such as DNA amplification.

Particularly, the chip itself may contain no valves for the control of liquids into the chambers. Accordingly, the chip may be very simple and contain no moving parts. This not only lowers the manufacturing costs for making the chip, but also enables a smaller chip structure with space for more chambers within a chip of a specific size. Further, it facilitates manufacturing of the device by use of a polymer and thereby enables high scale manufacturing, e.g. by injection moulding. Further, it may enable a very high robustness and reduce the risk of faults.

Particularly, the device may comprise, at each inlet, a diffusion barrier which is configured to prevent diffusion of macromolecules including genomic DNA and enzymes in or out of the reaction chambers. The diffusion barrier may further slow-down diffusion or reduce the diffusion time for ions whereby stoichiometric reaction becomes possible.

The hydraulic resistance at the diffusion barrier may e.g. be more than 100 times, or even more than 1000, more than 2000, more than 3000 or even more than 10000 times the resistance provided by the micro channels before and after the diffusion barrier.

The resistance may be symmetric about the diffusion barrier meaning that the resistance before the barrier equals the resistance after the barrier, or the resistance may be non-symmetric, e.g. with a larger resistance before the barrier than after the barrier, or with a larger resistance after the barrier than before the barrier.

The inlet may have a cross section with a circular, oval, square, or rectangular shape, or with other shapes. A largest dimension of the inlet, i.e. a largest dimension which can be measured in a cross section of the diffusion barrier may particularly be in the same order of magnitude as the persistence length of double stranded DNA. This is typically in the range of 10-200 nm such as in the range of 20-180 nm or in the range of 30-170 nm.

Advantageously, particularly if the device is made as an injection moulded chip of a polymer material, the chamber, micro channels, and the diffusion barrier are made as recesses in a surface of the chip, the recesses being covered by a cover layer. The diffusion barrier, in this embodiment, may particularly be a recess of a lower depth than the recesses constituting the micro channels and chambers.

If the chip is made from a polymer material, the micro channels, diffusion barriers, reaction chambers, and inlet/outlet structures are protrusions which in the fabrication process are formed in a mould surface which is subsequently used as a mould for imprinting the recesses in the chip.

The device may comprise a plurality of reagent delivery ports where each port has an associated delivery conduit extending from the delivery port to the common junction. Additional diffusion barriers may be located in the delivery conduits. The additional diffusion barriers may be similar to those at the inlets or they may have a larger or smaller cross section.

The reagent delivery ports are all on the same side of the chip, herein referred to as the upper side surface of the chip. This allows a simple and effective application of a control pressure to each port individually, e.g. by use of a so called chuck, facilitating control of fluid flow from the junction through each chamber individually without use of valves. Additionally, all outlets may also be on the upper side surface of the chip. Further, the common junction may comprise a common junction pressure port through which the pressure in the common junction can be controlled. The common junction pressure port may also be on the upper side surface of the chip.

The device may comprise a pressure control structure configured to apply control pressures at least at each outlet and at the common junction and/or at the reagent delivery ports. Particularly, the pressure control structure may be configured to control pressure individually at each delivery port.

In one embodiment, the device comprises a plurality of chambers, e.g. 2, 3, 4, 5 or more chambers, sharing a common junction via the micro channels. The chambers could be located in a circular layout, or a star layout about the common junction. In that way, the device takes up less space.

The reaction chambers, the inlets, the outlets, the pillars, and optionally, the common junction and micro channels may be formed in one piece. The device may e.g. include a chip or be constituted by a chip which is moulded, e.g. in one piece, e.g. in a polymeric material, e.g. a hydrophobic material or e.g. a material selected from a group consisting of: polypropylene, polyethylene, and amorphous polymer cyclic olefin copolymer materials.

To enable monitoring of the filling of the reaction chamber and mixing in the reaction chambers, the device may have at least one transparent or translucent wall section.

The device may further comprise a plurality of adjacent pillars in the reaction chambers, where each pillar forms its own axial direction extending from the bottom to the top, and where each pillar is separated from adjacent pillars by open slots.

Due to the very small size of the barrier, it becomes highly important that the walls of the chip do not deflect since such deflection may change the size of the barrier. By implementing the pillars, the rigidity can be increased, also when using polymer materials, and deflection of the walls of the chip can be prevented. This is particularly advantageous in combination with the diffusion barrier due to the need for narrow tolerances to preserve a specific resistance across the barrier.

Due to the pillars, the chip may be manufactured from less rigid materials, e.g. by injection moulding of polymer materials, e.g. thermoplastic materials. This may not only lower the manufacturing costs for making the chip, but it may also enable a smaller chip structure with space for more chambers within a chip of a specific size. More specifically, it may enable relatively wide chambers with long distances between the side walls. The distance between the side walls may particularly be above 1/1000 of the largest dimension of the device.

For the purpose of mixing the liquid reagents, a change in the flow direction of the reagent away from the straight direction from the inlet to the outlet may be desirable. For this purpose, the pillars may particularly be arranged to form at least one row extending in a row-direction which may particularly be transverse to the straight-line path from the inlet to the outlet.

Herein, the straight-line flow path is defined as a path in the direction along the shortest possible line from the inlet to the outlet.

By arranging the pillars in rows, the rows may provide phase guiding of the liquid reagents.

Molecules at an interface of a liquid will be in a higher energy state due to missing neighbours resulting in surface tension. This is given by Gibbs energy, G, per area, A

The fluid will always minimize the energy by minimizing the surface area—also when a pressure is applied to the reaction chamber and the fluid fills the chamber. The fluid will be pinched between the pillars are arranged in rows or in a rectangular lattice or in other patterns, the change in curvature of the interface per filled volume will be at a minimum when the fluid are filling the chamber along a row of pillars since the fluid will be pinched between two adjacent pillars. Due to the applied pressure the fluid, begins to wet around the pillars, and forms a characteristic contact angle to the substrate, not counting a change in angle due to applied pressure. The fluid forms an interface to the next pillar first when the fluid from the previous row contacts the propagating interface.

The pillars may form a contact angle of at least 30 degrees, or at least 40 degrees or at least 50 or at least 60 or at least 70 or at least 80 degrees to a fluid containing human DNA, e.g. to a diluted DNA solution containing TBE buffer, BME and Triton.

The pillars may be formed by a polymer material, particularly a polymer material selected from the group consisting of polypropylene, polyethylene, and amorphous polymer cyclic olefin copolymer materials (COC—also called Topas). In this case, the contact angle may particularly be in the range of 35-45 degrees to a fluid containing human DNA or the contact angle may particularly be above 90 degrees to water.

In this embodiment, the pillars and possibly also the bottom and top may preferably be made from one or more materials selected from this group.

Particularly, the pillars could be made from a hydrophobic material, e.g. a material selected from the above group and particularly from a material having a contact angle of at least 35 degrees to a fluid containing human DNA or forming a contact angle above 90 degrees to water.

The pillars in the reaction chamber thereby have two functions, namely that of preventing collapse of the bottom and top and that of providing phase guiding along the row.

Particularly, the device may comprise at least two, e.g. three, four, five, six, or more rows of pillars thereby forming an ordered array of pillars.

Adjacent pillars in a row may be spaced at most a first distance, and adjacent rows could be spaced at least a second distance being larger than the first distance. This will create rows of adjacent pillars which may form phase guiding of a meniscus in the reaction chamber back and forth along each adjacent row. In that way, the meniscus may follow the direction of the rows, i.e. the meniscus may follow a flow path comprising flow sections in different directions while the subsequent liquid may be allowed to pass through the spacing between adjacent pillars in a row of pillars and thereby follow the straight-line flow path. As a result, the meniscus and the subsequent flow may obtain different directions whereby the mixing capability of the chamber is increased.

To increase rigidity and strength of the chip further, at least one of the pillars may connect the bottom wall to the top wall of the chamber. The pillars and top and bottom walls may e.g. be moulded into the bottom and top walls or adhesively bonded thereto.

The slots, in a cross section perpendicular to the axial direction of the pillars, are smaller than the pillars.

The pillars may have a non-circular shape in a cross section perpendicular to the axial direction, e.g. an elliptic shape or an eye-shape, i.e. essentially the shape of a human eye—i.e a circular shape with at least one sharp pointed edges in a direction, and preferably with two sharp pointed edges in opposite directions.

Such shapes may provide an increased guiding effect for the meniscus since it will require a larger surface of the fluid to move around an edge when the edge has a smaller radius—i.e. when the edge is sharp. Accordingly, it will require a larger surface to move around the sharp pointed edge of the pillar

Adjacent pillars may be parallel or non-parallel, and the rows may likewise be parallel or non-parallel. Particularly, the rows may be non-parallel such that the aforementioned flow sections formed by adjacent rows widens out in the direction from the inlet towards the outlet.

In addition to the effect of limiting the transport of macromolecules through the diffusion barrier by the narrow geometry of the barrier, an optically actuated valve-effect can be enhanced by local heating of the fluid at diffusion barrier. This can be done in two ways using local heating: (i) local heating induces a temperature gradient. Particles and molecules can migrate in the temperature gradient (an effect known as thermo-phoresis) thus that diffusion of macromolecules through the heated area is hindered and (ii) a phase transition can be induced by heating for example by mixing a hydrogel polymer into the fluid that then creates a gel under local heating effectively creating a plug thus hindering the liquid to flow. For that purpose, the microfluidic device may include thermal means located to increase temperature of the liquid reagents locally at the diffusion barrier. Herein, thermal means is understood as any means for heating the fluid locally at the diffusion barrier, e.g. including light emitting means, microwave means, or other means for heating the fluid.

In a second aspect, the invention provides a method of mixing liquid reagents by use of a device as described above. The method comprises the steps of providing a flow of the liquid reagents through the reaction chamber, where the flow speed is adjusted by use of pressure control such that single DNA molecules are allowed to cross the diffusion barrier individually. Optionally, the reagents are mixed in the reaction chambers by use of the pillars. For this purpose, the pressure may be adjusted until phase guiding is achieved by the rows of pillars.

Referring to the above mentioned phase transition which can be induced by heating, the method may include the step of mixing a hydrogel polymer into the liquid reagent to thereby create a gel under local heating to thereby provide reduced liquid flow through the diffusion barrier.

### BRIEF DESCRIPTION OF DRAWINGS

In the following, embodiments of the invention will be described by way of example with reference to the figures in which:

FIG. 1 illustrates a chip device according to the invention;

FIG. 2 illustrates schematically two chambers of a device;

FIGS. 3a-3e illustrate the basic function of the device;

FIG. 4 illustrates that the device comprises a plurality of adjacent pillars;

FIGS. 5, 6a, 6b, 6c illustrate pillars for guiding the meniscus transverse to the straight-line path in the chamber;

FIGS. 7a and 7b illustrate fabrication steps;

FIG. 8 illustrates two images of imprints;

FIG. 9a illustrates a microscope image showing an imprint carried out in a CNI tool;

FIG. 9b illustrates a difference in manufacturing time when using different tools;

FIG. 10 relates to an experiment and shows filling of DNA into the device;

FIGS. 11a and 11b illustrate results from an off-chip amplification and negative control.

FIG. 12 illustrates details of the diffusion barrier; and

FIGS. 13a, 13b, 13c illustrates results of a simulation.

### DETAILED DESCRIPTION

Further scope of applicability of the present invention will become apparent from the following detailed description and specific examples. However, it should be understood that the detailed description and specific examples, while indicating preferred embodiments of the invention, are given by way of illustration only, since various changes and modifications within the scope of the invention will become apparent to those skilled in the art from this detailed description.

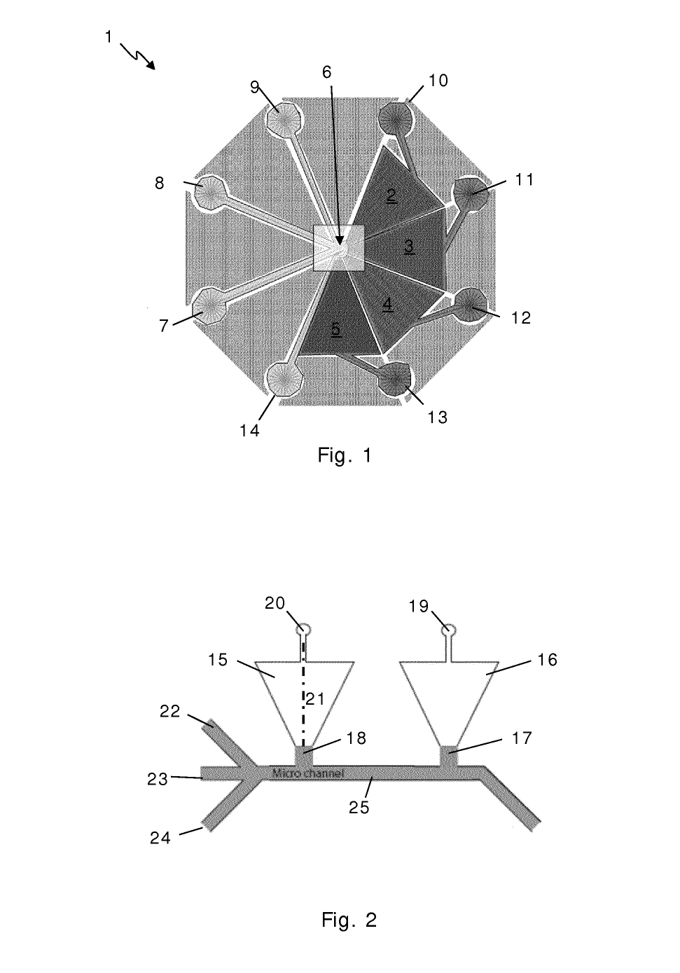

FIG. 1 illustrates a microfluidic device according to the invention. The device comprises a chip 1 forming 4 reaction chambers 2, 3, 4, 5.

Each reaction chamber extends between an inlet which is directly adjacent a common junction 6. The common junction 6 communicates with 3 intakes 7, 8, 9 for receiving different reagents. Each chamber further has an outlet 10, 11, 12, 13 for discharging the mixed reagents.

The chip 1 further forms a waste discharge 14 for discharging reagents which are not received in a chamber.

FIG. 2 illustrates two chambers 15, 16 arranged side by side. Each chamber has an inlet 17, 18 and an outlet 19, 20. In FIG. 2 it is illustrated that each chamber thereby forms a straight-line flow path indicated by the dotted line 21 from the inlet to the outlet.

The liquid reagents are added to the intakes 22, 23, 24 and conducted to the chambers via the common junction 25 by controlling pressure differences at the intakes, and outlets, optionally also at the common junction and/or at the inlets. The inlets comprise diffusion barriers for preventing diffusion of molecules such as DNA molecules.

To control the pressure differences, the chip could be mounted on a chuck with O-rings placed between the intakes, outlets and waste discharge and the chuck to ensure no leakage. Air hoses may connect the chuck to a pressure control device such as MFCS-FLEX, Fluigent where the pressure at a plurality of different channels can be adjusted independent of each other.

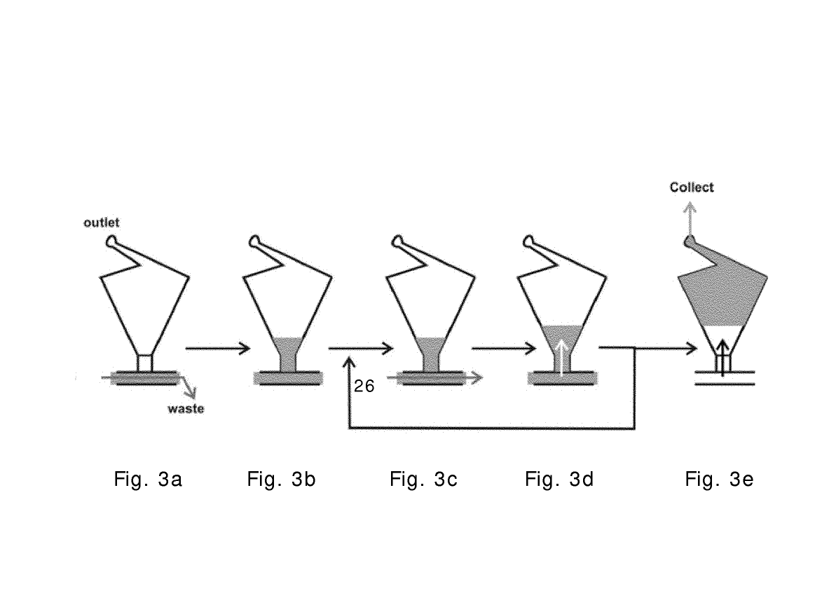

FIGS. 3a-3e illustrate the basic function of the device, i.e. on-chip amplification of DNA. In FIG. 3a, the sample is introduced by a pressure driven flow into the common junction. In FIG. 3b, a finite volume of the sample is, by pressure, introduced into the reaction chamber. In FIG. 3c, a reagent is, by pressure, forced into the bus. In FIG. 3d, a finite volume of the denaturation reagent is filled into the reaction chamber. The arrow 26 indicates that the steps of FIGS. 3c and 3d are repeated in order to introduce neutralisation and afterwards the solution which amplifies the sample. In FIG. 3e, the sample is amplified, and is forced into the outlet and collected e.g. by pipetting.

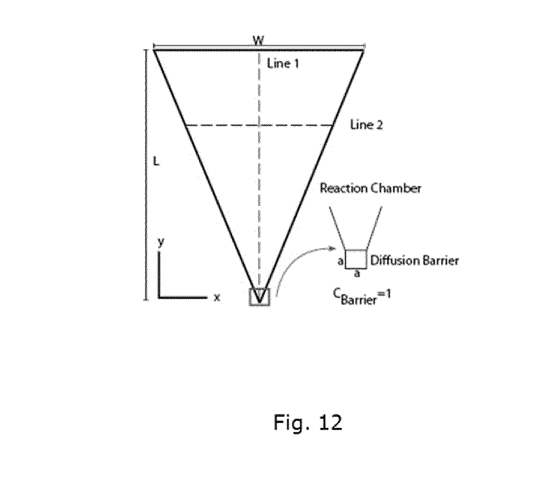

The diffusion barriers are design and fabricated with a restriction in the height. The diffusion will be limited due to a restriction dimension of the barrier height. The diffusion barrier will be described in further details with reference to FIG. 12.

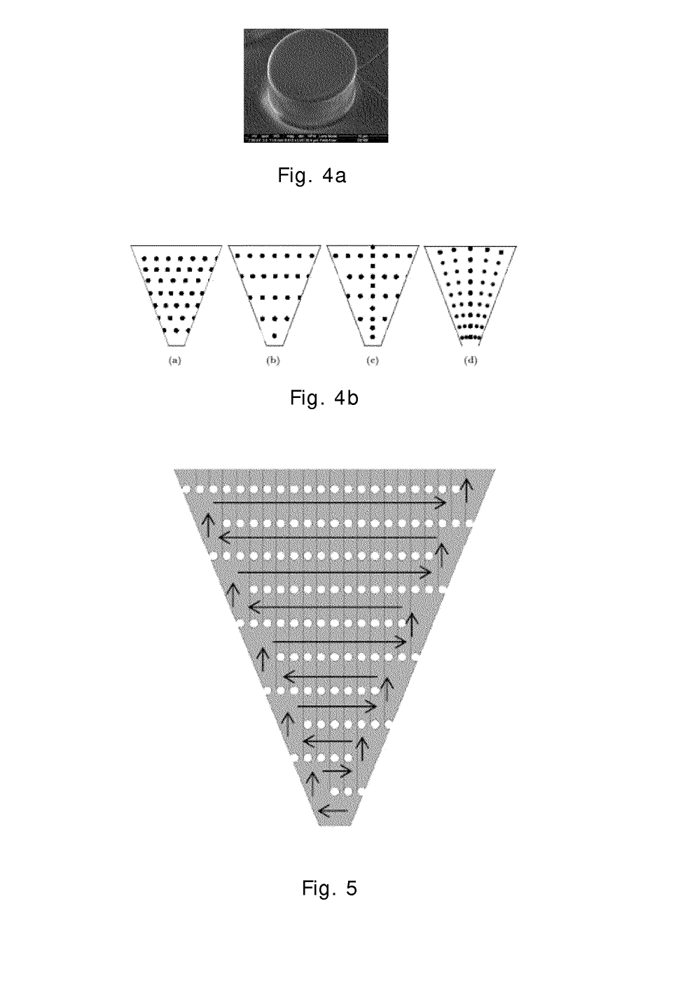

FIG. 4a illustrates a single pillar extending from the bottom of the chip. FIG. 4b illustrates that the device comprises a plurality of adjacent pillars extending from the bottom to the top of the chamber. Each pillar is separated from adjacent pillars by open slots.

The pillars may particularly be arranged in ordered array which thereby introduces phase guiding of fluid while maintaining a low path length for diffusion, thus ensuring proper mixing in the reaction chamber.

Particularly, FIG. 4 illustrates the four different layouts of the pillars in the reaction chamber. (a) The hexagonal design facilitated a direction independent filling rate. (b) The rectangular arranged pillar design facilitating transverse filling. (c) The rectangular pillar arrangement with a row of pillars in the middle of the chamber and (d) where the distance between the pillars are varied by having a spherical layout.

The fluid will try to minimize the energy by minimizing the surface area even when pressure is applied to the reaction chamber and the fluid fills the chamber. The fluid will be pinched between the pillars during filling. Clearly, the distance between the pillars determines the phase guiding, and according to the invention, the device may include a plurality of rows where adjacent pillars in a row are spaced a shorter distance than the shortest spacing between pillars of different rows.

FIG. 5 illustrates a preferred layout of pillars forming rows of adjacent pillars which form phase guiding of a meniscus in the reaction chamber back and forth along each adjacent row, illustrated by the arrows. In that way, the meniscus follow the direction of the rows, namely a flow path comprising flow sections in different directions while the subsequent liquid may be allowed to pass through the spacing between adjacent pillars in a row of pillars and thereby follow the straight-line flow path.

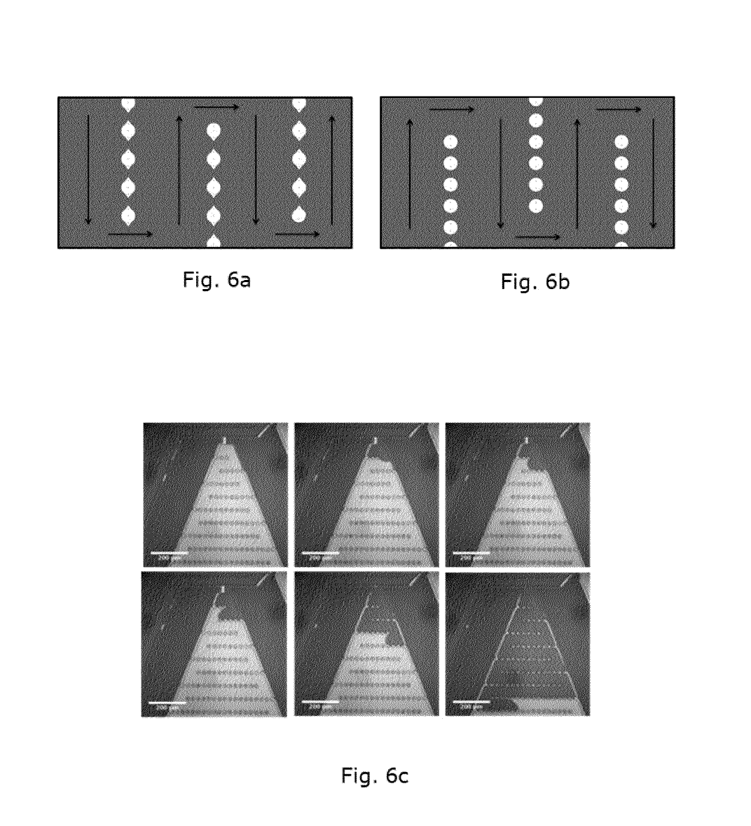

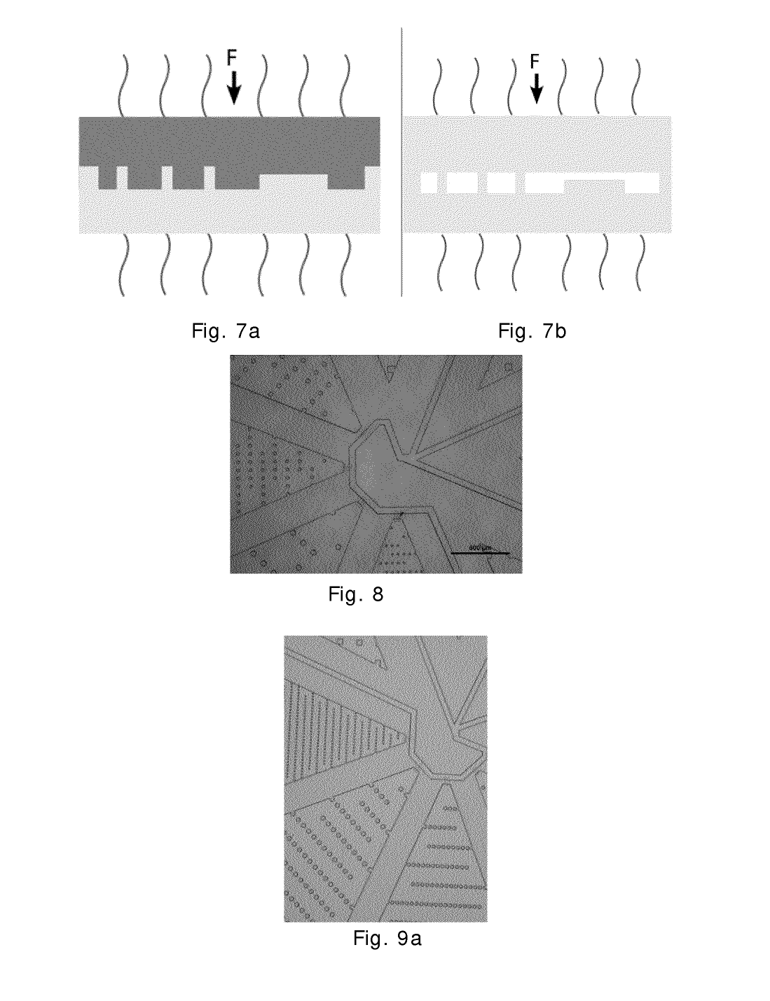

In FIG. 6a, the pillars have a cross sectional shape like an eye or a lemon, i.e. a circular shape with two sharp pointed edges in opposite directions. In FIG. 6b, the pillars have a circular cross sectional shape and in FIG. 6c, a photo is taken during an experiment. The photo clearly illustrates the phase guiding.

The chamber including the pillars may be treated with a buffer solution including Triton-X, e.g. in a concentration of 0.001% may reduce sticking of the biological material to the surfaces of the device.

The fabrication of the chip may be carried out as a two-step process in which a stamp is firstly thermally imprinted in polymer to achieve the desired form, c.f. FIG. 7a, and subsequently, the structured polymer and a planar polymer sheet are thermal bonded c.f. FIG. 7b.

The imprint material could be transparent and should be suitable for imprint. The amorphous polymer cyclic olefin copolymer (COC), also called Topas, is a suitable material for injection moulding.

FIG. 8 illustrates two images of imprint done with an applied pressure of 10 kN, imprint temperature at 190° C. and de-moulding at 70° C. (a) Shows a 10 min imprint where filling of the cavity at the centre is not completed. (b) An imprint time of 20 min shows on the other hand perfect imprint result.

By using a thermal imprint machine in which the thermal mass is relatively low, the imprint protocol may be faster. The imprint could be made e.g. in a dual-use tool designed for optical double-side lithography and precision alignment, e.g. from the company EVG. For wafer sizes up to 150 mm EVG 620 could be used.

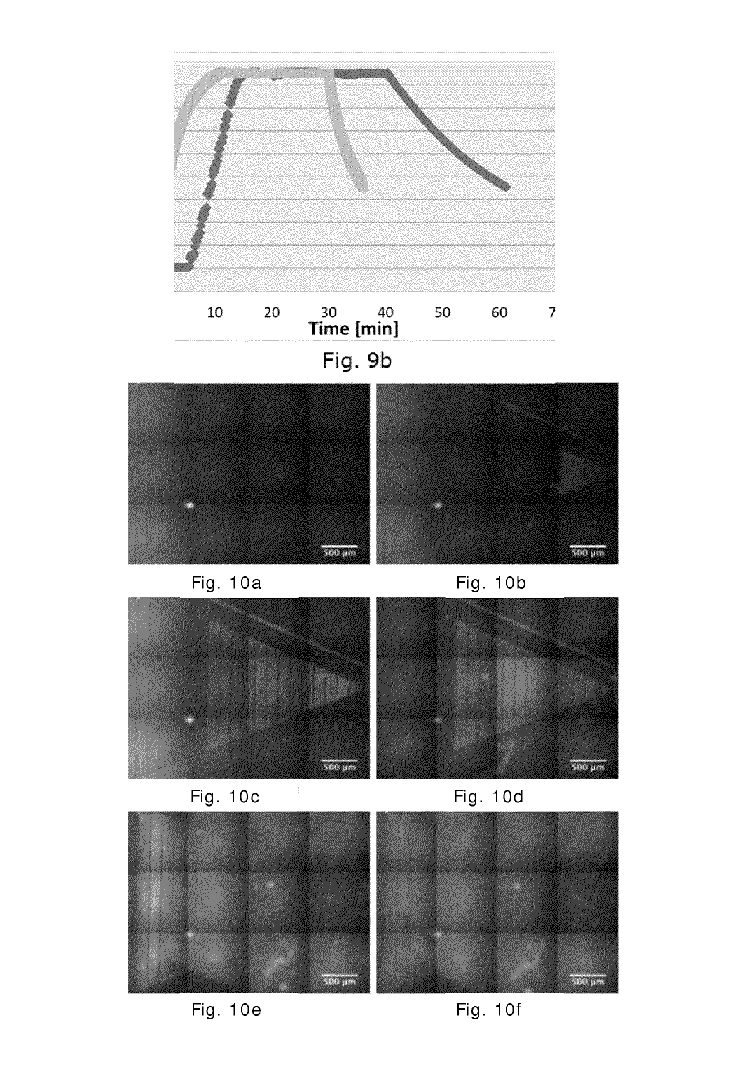

FIG. 9a is a microscope image showing an imprint carried out in CNI. (b)The plot compares the thermal cycle of a 20 min imprint done at 190° C. in an EVG620 tool and CNI tool from a company called NIL Technology. By imprinting in the CNI instead of EVG620, the thermal cycle is reduced to 24 min.

The chambers are located in a circular layout or star layout about a common junction. The inlets are all connected to the common junction via micro channels, and intake leads different reagents to the junction. As previously mentioned, the fluid flow is controlled by pressure differences.

Experiment

A protocol to prevent or minimize DNA to stick to the walls was developed before carrying out the amplification. The first step in the MDA protocol was to introduce the double stranded λ-DNA into the chip. This was done by pipetting 5 μl of the diluted DNA solution containing TBE buffer, BME and Triton into the ‘waste’ reservoir. A pressure of 200 mbar was applied to the channel connected to ‘waste’ thus forcing the liquid into the bus and filling all the way to inlet one, two and three to three of the chambers. By doing so, air bubbles were avoided in the bus and the DNA material that was not filled into the reactions chambers was kept in the waste reservoir. During the filling of the bus, a pressure of 400 mbar was applied to the backside of the reaction chambers to ensure that an unwanted burst of sample materials into the reaction chambers would not occur.

The sample containing the DNA was filled into the chamber by applying a pressure to the bus as described in the previous chapter. After bursting into the chamber, the pressure was decreased to avoid a too fast filing rate which would risk unwanted trapping of air bubbles. From the provided protocol is it known that the stoichiometric reaction should be mixed with following volumes: 1:1:2:4

The filling of DNA is shown in FIGS. 10a-10c where an imaging procedure was carried out with 4×3 frames of the chamber.

The filling of DNA is shown in FIGS. 10a-10c where an imaging procedure was carried out with 4×3 frames of the chamber. In FIG. 10a time t=0 minutes, temperature is 20° C., in FIG. 10b t=1 min, temperature is 20° C. in FIG. 10c t=15 min, temperature is 20° C., in FIG. 10d t=15 min, temperature is 20° C., in FIG. 10e t=30 min, temperature is 20° C., and in FIG. 10f t=40 min, temperature is 45° C.

The frames were afterwards stitched using MATLAB. A heat cartridge is placed in contact with the chip, c.f. FIG. 10d. This allow for fast and accurate temperature control. It is clear that the background fluorescence changes when the cartridge is placed on the chip. Due to this, it is not possible to subtract the background from the frames.

FIG. 10e shows ⅙ of the reaction chamber filled with the denaturation solution. The DNA was at this stage not denatured, which is seen in the left part of the image.

In FIG. 10f the chip heated to 45° C. It is seen that the fluorescence is decreased in the left part of the chamber. This effect occurred instantly but the temperature was held constant for 10 min in order to denature the strands into ssDNA and all base pair bonds broken. Also the fluorescence of YOYO is temperature dependent, thus the chip was cooled down and if the fluorescence intensity stayed low it was concluded that the DNA was denatured.

The next step in the protocol is the neutralization step where a buffer neutralizing the pH is introduced. 0:01% of triton was added to the stop solution and the chamber was filled with a volume of ⅙ of the total volume.

The final step is to measure the concentration of DNA collected from the outlet. This was done by an ethanol precipitation and spectrophotometric measurement.

Due to the very low amount of sample in the reservoir (a volume 5-20%) a low DNA concentration was expected when 5 μl of milliQ water was pipetted into the outlet reservoir and afterwards collected. The sample was according to the protocol heated to 65° C. to inactivate the polymerase. Before measuring the concentration of the amplified sample, an ethanol precipitation was carried out. Ethanol precipitation is a purification tool removing the nucleotides and salt from the sample and an added benefit is an increased concentration of the sample. After collecting the sample, 2:5 μl of 5M ammonium acetate and 5 μl of 99:9% was added and mixed by pipetting. The mixture was kept at −20° C. overnight and the next day placed in a micro centrifuge at 4° C. and spun for 30 min at 14000 rpm. Typically, for purification of DNA, a small pellet containing the DNA is found at the bottom of the micro tube, but due to the very low amount of DNA was this invisible. The hinge portion of the tube was therefore oriented towards the centre of the micro centrifuge thus knowing where to expect the pellet. The supernatant was carefully pipped out of the chip thus leaving the DNA in 1-2 μl which was air dried. Finally, 2 μl of MilliQ water was added thus a 2.5 times higher concentration compared to the 5 μl was used to collect the DNA form the reservoir.

In order to determine the average concentration of the amplified ssDNA, a spectrophotometric analysis was performed. A spectrophotometric measurement of the MDA protocol of the collected material, a measurement of a MDA sample done off-chip with the identical protocol, and a measurement where the stating sample was without λ DNA but otherwise following the MDA protocol (negative amplification) were carried out.

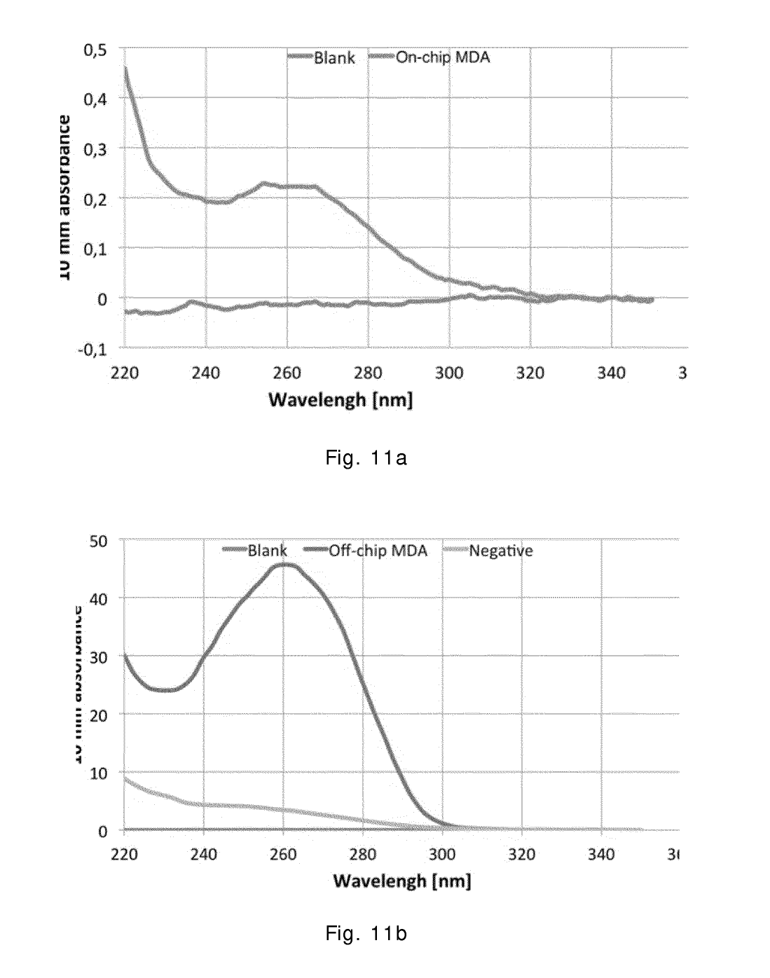

All measurement was done by pipetting 1 μl sample to the pedestal. The results from the off-chip amplification and the negative control are seen in FIG. 11 where the absorbance from the off-chip amplification shows a typical absorbance spectrum for DNA.

Details about the Diffusion Barrier

To understand the diffusion in the fluids, we refer to Fick's laws of diffusion. The diffusion barriers according to the invention may particularly be made as a restriction of the height of the imprint in a polymer chip.

In the amplification process, the DNA sample is filled in reaction chamber with a finite volume and the denaturation reagent is afterwards introduced into the bus.

The reagent has a diffusion constant of D=2*10⁻⁹ m²/s corresponding to small ions.

When a pressure of only ΔP=1 mbar is applied to the bus, the fluid will flow.

The flow of an incompressible Newtonian fluid through a straight channel driven by an applied pressure, ΔP is given by:

ΔP=R_{hyd}Q

where Q is the flow rate and the hydraulic resistance R_{hyd} of a fluid in a channel with a rectangular cross-section with the width, w, the height, h, and length, L_{bus}, is given by:

$${R}_{hyd} = \frac{12 η {L}_{bus}}{1 - 0.63 ( \frac{h}{w} )} \frac{1}{{h}^{3} w}$$

where η=1 mPa, s is the viscosity of water at 20° C.

The exchange time for e.g. a 50*50*30 μm volume in front of the diffusion barrier can be calculated as:

$$t = \frac{Q}{V} = \frac{\frac{Δ P}{{R}_{hyd}}}{w h a} = 9.3 s$$

When the volume in front of the diffusion barrier is exchanged each 9.3 sekund it is clear that the concentration at the entry should be fixed at C_{barrier}=1 a.u. Diffusion through the area separating the bus and the reaction chamber is simulated by solving the geometry shown in FIG. 12 The concentration is arbitrary and set to c_{chamber}=0 in the chamber and c_{bus}=1 in the bus at time t=0. In this simulation, there is conservation of reagent and no convection. The volume of the fluid in the chamber is ⅛ of the total volume, and the ratio between the L and W is kept as in FIG. 12

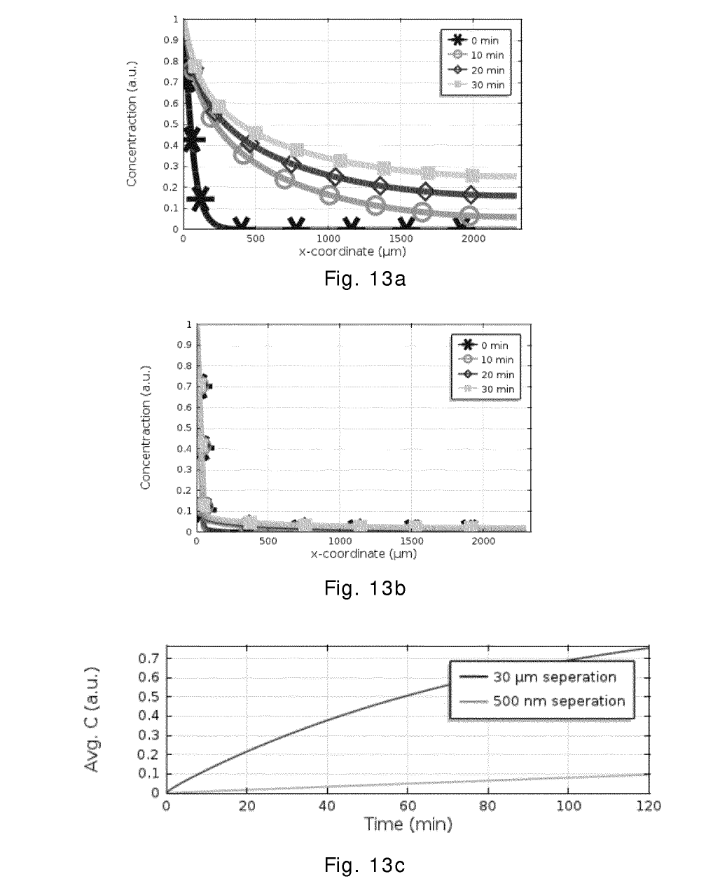

The result of this simulation is shown in FIG. 13a where the concentration is plotted along the middle of the chamber (line 1 in the COMSOL sketch in FIG. 12) for a diffusion barrier with the same height as the reaction chamber (30 μm). In FIG. 13b, the height of the separation is reduced to 500 nm such that the reaction chamber is kept at 30 μm. It is clear from FIGS. 12 and 13 that the confinement from the nano slit at the inlet is an effective diffusion barrier. It is possible to handle different reagents in the bus by applying the diffusion barrier to the chip layout, and thus avoid ‘contamination’ into the reaction chamber. This is important to avoid (or at least restrict) the diffusion into the chamber due to the strict stoichiometric MDA protocol. In FIG. 13c the average concentration in the chamber as a function for the two barriers is plotted, and it is observed that the average concentration is only 5% of the concentration in the bus after one hour while the average concentration without the restriction is 50%. The differences in concentration show that the chosen design solution is an effective barrier for the diffusion allowing the stoichiometric protocol to be carried out.

### List of Embodiments

1. A microfluidic device for macromolecule amplification by sequential addition of liquid reagents, the device comprising, a chip forming a plurality of reaction chambers each extending between an inlet and an outlet, each inlet being in fluid communication with a common junction via micro channels, characterised in that the device comprises a diffusion barrier at each inlet configured to increase the pressure threshold for a reagent to cross the resistor.

2. A device according to embodiment 1, where each resistor is configured to prevent diffusion of macromolecules including genomic DNA and enzymes in or out of the reaction chambers.

3. A device according to any of the preceding embodiments, where the diffusion barrier has a largest dimension in the range of 10-200 nm.

4. A device according to any of the preceding embodiments, where the chamber, micro channels, and the diffusion barrier are constituted by recesses in a surface of the chip, the recess constituting the diffusion barrier having a lower depth than the recesses constituting the micro channels and chambers.

5. A device according to embodiment 4, where the recesses are covered by a cover layer bonded to the surface.

6. A device according to any of the preceding embodiments, comprising a plurality of reagent delivery ports where each port has an associated delivery conduit extending from the delivery port to the common junction.

7. A device according to embodiment 6, where the reagent delivery ports are all on the same side of the chip.

8. A device according to any of the preceding embodiments, further comprising a pressure control structure configured to apply control pressures at least at each outlet and at the common junction.

9. A device according to embodiment 8, where the pressure control structure is configured to control pressure individually at each delivery port.

10. A device according to any of the preceding embodiments, where the chambers are located in a circular layout about the common junction.

11. A device according to any of the preceding embodiments, wherein the chip forms the reaction chambers, the inlets, the outlets, the common junction, and optionally the ports and the delivery conduits in one piece.

12. A device according to embodiment 11, where the chip is moulded in a polymeric material.

13. A device according to any of the preceding embodiments, where each chamber has at least one transparent or translucent wall section.

14. A device according to any of the preceding embodiments, further comprising a plurality of adjacent pillars in the reaction chambers, where each pillar forms its own axial direction extending from the bottom to the top, and where each pillar is separated from adjacent pillars by open slots.

15. A device according to embodiment 14, where the pillars are arranged to form at least one row extending in a row-direction being transverse to the straight-line flow path.

16. A device according to embodiment 14 or 15, comprising at least two rows each comprising a plurality of adjacent pillars, where adjacent pillars in a row are spaced at most a first distance, and where adjacent rows are spaced at least a second distance, the second distance being larger than the first distance.

17. A device according to any of the preceding embodiments, where at least one of the pillars connects a bottom wall to a top wall of the chamber.

18. A device according to any of embodiments 14-17, where the slots, in a cross section perpendicular to the axial direction of the pillars, are smaller than the pillars.

19. A device according to any of the preceding embodiments, further comprising thermal means located to increase the temperature of the liquid reagents locally at the diffusion barrier.

20. A method of mixing liquid reagents by use of a device according to any of embodiments 1-19s, the method comprising providing a flow of the liquid reagents through the reaction chamber, where the flow speed is adjusted by use of pressure control such that single DNA molecules are allowed to cross the diffusion barrier individually.

21. A method according to embodiment 20, comprising mixing a hydrogel polymer into the liquid reagent and use local heating to thereby create a gel to reduced liquid flow through the diffusion barrier.

1. A microfluidic device for macromolecule amplification by sequential addition of liquid reagents, the device comprising, a chip forming a plurality of reaction chambers each extending between an inlet and an outlet, each inlet being in fluid communication with a common junction via micro channels, characterised in that the device comprises a diffusion barrier at each inlet configured to increase the pressure threshold for a reagent to cross the diffusion barrier.

2. The device according to claim 1, where each diffusion barrier is configured to prevent diffusion of macromolecules including genomic DNA and enzymes in or out of the reaction chambers.

3. The device according to claim 1, where the diffusion barrier has a largest dimension in the range of 10-500 nm.

4. The device according to claim 1, where the chamber, micro channels, and the diffusion barrier are constituted by recesses in a surface of the chip, the recesses constituting the diffusion barrier having a lower depth than the recesses constituting the micro channels and chambers.

5. The device according to claim 4, where the recesses are covered by a cover layer bonded to the surface.

6. The device according to claim 1, comprising a plurality of reagent delivery ports where each port has an associated delivery conduit extending from the delivery port to the common junction.

7. The device according to claim 6, where the reagent delivery ports are all on the same side of the chip.

8. The device according to claim 1, further comprising a pressure control structure configured to apply control pressures at least at each outlet and at the common junction.

9. The device according to claim 8, where the pressure control structure is configured to control pressure individually at each delivery port.

10. The device according to claim 1, where the chambers are located in a circular layout about the common junction.

11. The device according to claim 1, wherein the chip forms the reaction chambers, the inlets, the outlets, the common junction, and optionally the ports and the delivery conduits in one piece.

12. The device according to claim 1, where at least the diffusion barrier is made from a material selected from the group consisting of polypropylene, polyethylene, and amorphous polymer cyclic olefin copolymer materials.

13. The device according to claim 1, where the chip is moulded in a material selected from the group consisting of polypropylene, polyethylene, and amorphous polymer cyclic olefin copolymer materials.

14. The device according to claim 1, where each chamber has at least one transparent or translucent wall section.

15. A method of mixing liquid reagents by use of a device according to claim 1, the method comprising providing a flow of the liquid reagents through the reaction chamber, where the flow speed is adjusted by use of pressure control such that single DNA molecules are allowed to cross the diffusion barrier individually.

16. The method according to claim 15, comprising mixing a hydrogel polymer into the liquid reagent and use local heating to thereby create a gel to reduced liquid flow through the diffusion barrier.
